(19) United States
(12) Patent Application Publication
(10) Pub. No.: US 2020/0330688 A1
(43) Pub. Date: Oct. 22, 2020

(54) SYRINGE WITH BACKPRESSURE PREVENTER AND ASSOCIATED METHODS

(71) Applicant: Merit Medical Systems, Inc., South Jordan, UT (US)

(72) Inventors: David W. Johnson, South Jordan, UT (US); Sarah Ashley Spangler, Midvale, UT (US)

(21) Appl. No.: 16/852,105

(22) Filed: Apr. 17, 2020

Related U.S. Application Data
(60) Provisional application No. 62/836,440, filed on Apr. 19, 2019.

(51) Int. Cl. A61M 5/315; A61M 5/31

(52) U.S. Cl. CPC A61M 5/31505; A61M 2205/582; A61M 2205/581; A61M 5/3129

(57) ABSTRACT

Devices used to deliver a medical fluid and/or medicament are disclosed herein. The devices may be configured to deliver an accurate volume of fluid and/or medicament. The devices may include a syringe having a barrel member and a plunger member comprising a detent to couple with a retention member of the barrel member to restrict retraction of the plunger member from a locked state due to backpressure.

### RELATED APPLICATIONS

This application claims priority to U.S. Provisional Application No. 62/836,440, filed on Apr. 19, 2019 and titled, “SYRINGE WITH BACKPRESSURE PREVENTER AND ASSOCIATED METHODS,” which is hereby incorporated by reference in its entirety.

### TECHNICAL FIELD

The present disclosure relates to medical instruments and systems for transferring a medicament. More specifically, in some embodiments, the present disclosure relates to a syringe configured to displace fluids such a fluid medicaments. The features relating to the methods and devices described herein can be applied to any syringe used to deliver a fluid and/or medicament.

### BRIEF DESCRIPTION OF THE DRAWINGS

The embodiments disclosed herein will become more fully apparent from the following description and appended claims, taken in conjunction with the accompanying drawings. The drawings depict only typical embodiments, which embodiments will be described with additional specificity and detail in connection with the drawings in which:

FIG. 1 is a perspective view of an embodiment of a syringe.

FIG. 2A is a cross-sectional side view of a barrel member of the syringe of FIG. 1.

FIG. 2B is a cross-section detail view of a proximal portion of the barrel member of FIG. 2A.

FIG. 3 is a side view of a plunger member of the syringe of FIG. 1.

FIG. 4A is a cross-sectional side view of the syringe of FIG. 1 in a plunger unlocked state.

FIG. 4B is a cross-sectional side view of the syringe of FIG. 1 in a plunger locked state.

### DETAILED DESCRIPTION

Medical procedures which include delivery of a fluid and/or a medicament to a patient are performed in hospitals, outpatient clinics, and in some instances outside of medical facilities, such as at a patient's home. Such procedures may be performed to provide therapeutic treatment including delivery of an accurate quantity of the fluid and/or medicament. Delivery of an inaccurate quantity of the fluid and/or medicament may be considered a medical error and lead to complications. Delivery of a fluid and/or a medicament may entail the use of needles, syringes, fluid pumps, fluid delivery lines, and so forth. In certain circumstances, a backpressure may be directed into the syringe when the fluid and/or the medicament is delivered into a secondary apparatus such as a fluid pump or delivery line or when the fluid and/or medicament is administered to a patient. The backpressure may cause inadvertent backfilling of the syringe with the fluid and/or the medicament and may result in deliverance of an inaccurate or incorrect quantity of the fluid and/or medicament.

Embodiments of the disclosure may be understood by reference to the drawings, wherein like parts are designated by like numerals throughout. It will be understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to, or in communication with, each other even though they are not in direct contact with each other. For example, two components may be coupled to, or in communication with, each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As specifically applied to a syringe such as the syringe depicted in the figures, the proximal end of the syringe refers to the end nearest the flange, and the distal end refers to the opposite end, the end nearest the inlet/outlet port of the syringe.

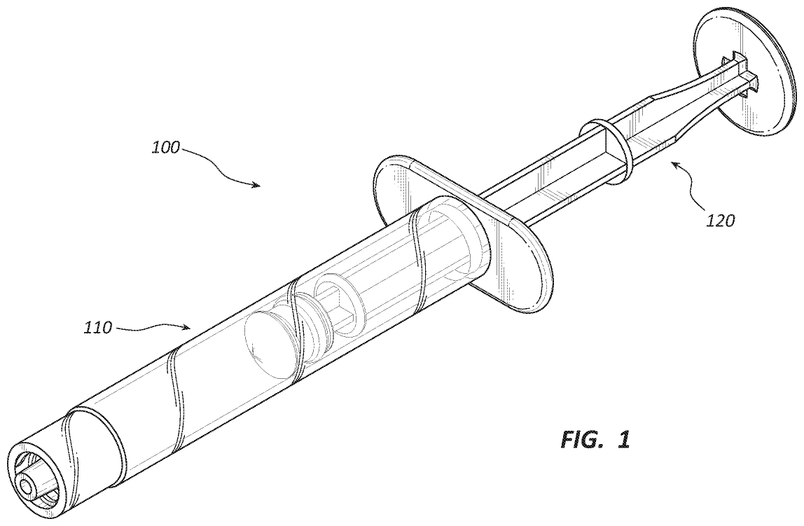

FIGS. 1-4B illustrate different views of an anti-backfill syringe and related components. In certain views each syringe may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

FIGS. 1-4B depict an embodiment of an anti-backfill syringe 100. In the illustrated embodiment, the syringe 100 comprises a barrel member 110 and a plunger member 120, in addition to other components.

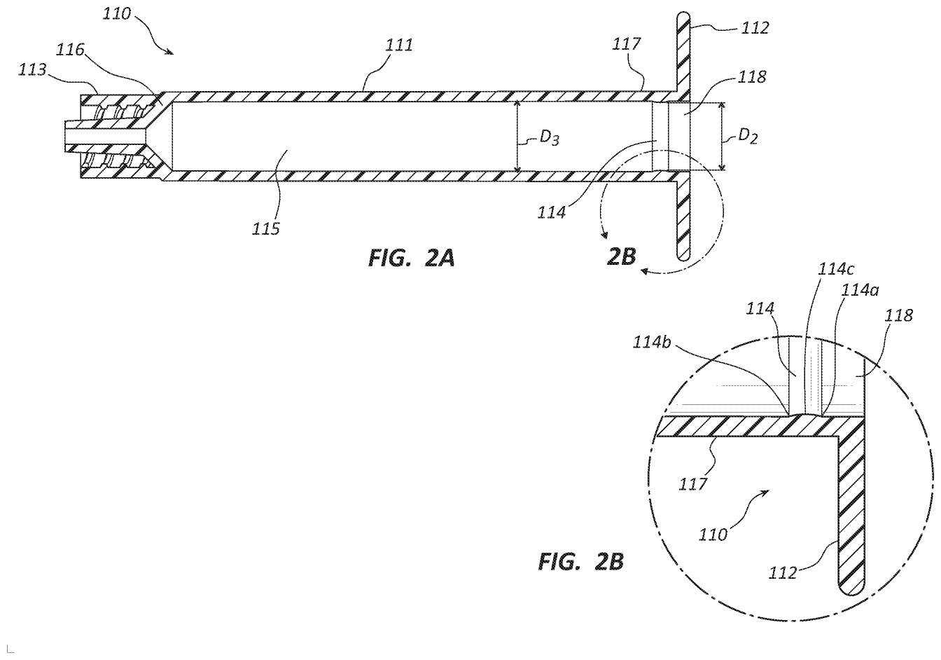

FIG. 2A is a cross-sectional view of the barrel member 110. As shown in FIG. 2A, the barrel member 110 of the illustrated embodiment comprises a barrel 111, a flange 112, a retention member or ring 114, and a port 113. The barrel 111 may be cylindrical in shape with a bore 115 which defines a reservoir for fluids or medicaments. Other barrel shapes are within the scope of this disclosure. In some embodiments, an injectable volume of the bore 115 may range from about 0.25 milliliter to about 100 milliliters, including from about 0.25 milliliter to about 75 milliliters and from about 0.25 milliliter to about 60 milliliters. In other embodiments, larger or smaller syringes may also be configured with elements of the present disclosure. The barrel member 110 may be formed from any suitable rigid or semi-rigid material, such as polycarbonate, polypropylene, polyethylene, glass, etc. The barrel member 110 may be manufactured using any suitable manufacturing technique, such as injection molding, casting, machining, etc.

As depicted in the illustrated embodiment, the barrel member 110 further comprises a proximal opening 118 disposed adjacent to a proximal end portion 117. The retention member 114 may be disposed adjacent the proximal opening 118. In other embodiments, the retention member 114 may be disposed at various locations within the barrel member 110. FIG. 2B is a detail cross-sectional view of the proximal end portion 117 of the barrel member 110. As depicted in FIG. 2B, in the illustrated embodiment, the retention member 114 protrudes radially inward into the bore 115 and is a continuous ring of material. In other embodiments, the retention member 114 may be a segment of a ring extending around a portion of the bore 115. In still other embodiments, the retention member 114 may be composed of a plurality of ring segments with gaps disposed between the ring segments. Furthermore, embodiments wherein the retention member 114 comprises protrusions, nubs, detents, indentations, ridges, grooves, or other geometries are likewise within the scope of this disclosure.

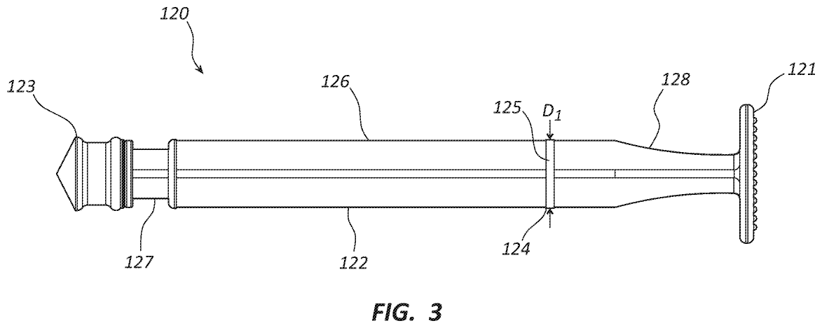

In the illustrated embodiment, the retention member 114 includes a proximal face 114a and a distal face 114b. The faces 114a, 114b may be radiused or sloped from a wall of the bore 115 toward a central portion 114c of the retention member 114. In certain embodiments, the slope angle may range from about 5 degrees to about 45 degrees, from about 5 degrees to about 30 degrees, or from about 5 degrees to about 15 degrees. The angle of the slope of the proximal face 114a may be substantially equivalent to the slope of the distal face 114b. This configuration may result in a substantially equivalent force to displace the plunger member 120 relative to the retention member 114 either proximally or distally when a detent or backstop (such as 124 of FIG. 3) engages the retention member 114. In some embodiments, the slope of either face 114a, 114b may be different from the slope of the other face 114a, 114b. For example, the slope of the distal face 114b may be greater than the slope of the proximal face 114a such that a force to displace the plunger member 120 proximally relative to the retention member 114 may be greater than a force to displace the plunger member 120 distally when the detent 124 engages the retention member 114.

The retention member 114 has a diameter D₂ as shown in FIG. 2A. The diameter D₂ is defined by a distance between two points on the central portion 114c, 180 degrees apart. In the illustrated embodiment, the diameter D₂ is smaller than a diameter D₃ of the bore 115 such that a detent (such as 124 of FIG. 3) may freely be displaced within the bore 115 without interfering with, or causing significant friction along, the wall of the bore 115. As D₂ is smaller than D_{3,} the retention member 114 is configured to create an interference with the detent (124 of FIG. 3). This interference is defined as a one-sided dimensional overlap between D₂ and D₃. A measurement of the overlap may vary depending on the materials used to form the retention member 114 and the detent (124 of FIG. 3). For example, when ABS or polycarbonate are used to form the retention member 114 and the detent (124 of FIG. 3), the interference may range from about 0.002 inch to about 0.100 inch, including from about 0.005 inch to about 0.050 inch and from about 0.010 inch to about 0.025 inch. When other materials are used, the overlap may be greater or less.

In the illustrated embodiment depicted in FIG. 2A, the flange 112 of the barrel member 110 is disposed adjacent the proximal end portion 117. The flange 112 may be configured to provide finger gripping surfaces. As shown in FIG. 2A, the flange 112 comprises two portions which extend radially outward from a central axis of the barrel 111. In some embodiments, the flange 112 may comprise a circular shape such that the flange 112 extends circumferentially radially outward from a central longitudinal axis of the barrel 111. The flange 112 may further comprise any suitable grip enhancing feature, such as ribs, dimples, detents, grooves, etc., disposed on a distally facing surface.

The port 113 is disposed adjacent a distal end portion 116 of the barrel member 110. In the illustrated embodiment, the port 113 is configured as a male Luer lock fitting having a Luer taper outer surface and a lumen that is in fluid communication with the bore 115 of the barrel 111. The port 113 may include a collar having internal threads configured to couple with external threads of a female Luer lock connector. Still further various port shapes, fittings, and connectors are within the scope of this disclosure.

In some embodiments, indices (not shown) may be disposed on an outer surface of the barrel 111. The indices may indicate the volume of the bore 115 in increments of milliliters, tenths of milliliters, hundredths of milliliters or any other suitable volume measurement. In other embodiments, the indices may indicate a quantity of a medicament in units, grams, milligrams, micrograms, etc. The indices may be applied to the barrel 111 using any suitable technique, such as transfer printing, laser printing, adhesive labels, cliché, etc. The indices may start at zero at the distal end portion 116 of the barrel member 110 and incrementally increase toward the proximal end portion 117.In other, embodiments, the indices may start at zero at the proximal end portion 117 and incrementally increase toward the distal end portion.

Referring to FIG. 3, a side view of the plunger member 120 is shown. The plunger member 120 is configured to be at least partly longitudinally disposed within the bore (115 of FIG. 2A) of the barrel (111 of FIG. 2A). In the illustrated embodiment, the plunger member 120 comprises a shaft 126, a plunger tip 123, a distal detent 129, and a detent or backstop 124. The shaft 126 comprises longitudinally oriented ribs 122. The shaft 126 may comprise any suitable number of ribs 122, such as two, three, four, etc. In some embodiments, the shaft 126 comprises a longitudinally extending cylindrical shaft. A plunger flange 121 is disposed adjacent a proximal end portion 128 of the shaft 126. The plunger flange 121 is configured to facilitate axial displacement of the plunger member 120 by a practitioner or individual. The plunger flange 121 may be shaped as a circular disk or any other suitable shape. The plunger flange 121 may be oriented perpendicular to a longitudinal axis of the shaft 126. The plunger flange 121 may comprise grip enhancing features on a proximally facing surface. In some embodiments the plunger flange 121 may be coupled to a handle, gripping ring, or other feature to facilitate gripping and displacement of the plunger member 120. The grip enhancing features may comprise ridges, grooves, dimples, detents, surface texturing, etc. The distal detent 129, may be configured to engage with the retention member (114 of FIG. 2A) to retain at least a portion of the plunger member 120 within the barrel member (110 of FIG. 2A). The shaft 126 may be formed from any suitable rigid or semi-rigid polymeric material, such as polycarbonate, polypropylene, polyethylene, ABS, etc. The shaft 126 may be manufactured using any suitable technique, such as injection molding, casting, machining, etc.

With continued reference to FIG. 3 as well as FIGS. 2A and 2B, a distal end portion 127 of the shaft 126 may be configured to be coupled to the plunger tip 123. The plunger tip 123 may be configured to seal against the wall of the bore 115 of the barrel 111 such that negative or positive gage pressure can be generated within the bore 115 distal to the plunger tip 123 when the plunger member 120 is displaced axially along the bore 115 of the barrel 111. Thus, displacement of the plunger member 120 and plunger tip 123 within the bore 115 of the barrel 111 may displace fluid by drawing the fluid into the bore 115 or ejecting fluid from the bore 115. The plunger tip 123 may be formed of any suitable elastomeric material, such as rubber, thermoplastic elastomers, etc. The plunger tip 123 may be manufactured using any suitable technique for elastomeric materials, such as injection molding, transfer molding, compression molding, etc. In other embodiments, the plunger tip 123 may be integrally formed with the shaft 126 and be configured as an integrated tip, wiper, etc.

The detent 124 may be coupled to the shaft 126 and disposed adjacent the proximal end portion 128 of the shaft 126. In other embodiments, the detent 124 may be disposed at any suitable position along the shaft 126. The detent 124, as depicted, comprises a flange 125. In other embodiments, the detent 124 may comprise a bump, a pin, a ramp, etc., disposed on the shaft 126. In some embodiments, the detent 124 may be integrally formed with the shaft 126, including embodiments wherein the detent 124, or any portion thereof, is a molded feature of the shaft 126. In other embodiments, the detent 124 may be a separate component that is coupled to the shaft 126 using any suitable technique, such as overmolding, heat welding, sonic welding, adhesive, etc. The detent 124 may be formed from any suitable rigid polymeric or semi-rigid material, such as polycarbonate, polypropylene, polyethylene, ABS, etc. In the embodiment of FIGS. 1-4B, the detent 124 is configured to engage with the retention member 114 when the plunger member 120 is proximally or distally displaced, such that interference between the retention member 114 and the detent 124 restricts displacement of the plunger member 120 with respect to the barrel member 110.

In the illustrated embodiment, the flange 125 extends radially outward from the longitudinal axis of the shaft 126. The flange 125 may be disposed between the ribs 122 of the shaft 126. In some embodiments, the detent 124 may be disposed between at least two of the ribs 122 such that the flange 125 forms an arc between the two ribs 122. In other embodiments, the detent 124 may be disposed between more than two ribs 122 such that the flange 125 forms a circumferential arc around the shaft 126.

In the illustrated embodiment, the outside diameter Di of the flange 125 is configured to be greater than inside diameter D₂ (FIG. 2A) of the retention member 114 and less than the inside diameter D3 (FIG. 2A) of the bore 115. Thus, the plunger member 120 may be slidably displaceable within the bore 115, without interference or drag caused by the detent 124, when the detent 124 is disposed proximal of the retention member 114. Further, the plunger member 120 may be restricted from displacement when the detent 124 is disposed distal of the retention member 114. In other words, once the detent 124 is advanced distally past the retention member 114, interaction between the retention member 114 and the detent 124 may resist proximal displacement of the plunger member 120 with respect to the bore 115. An outer edge of the flange 125 may be radiused or squared or have any other suitable shape or geometry configured to engage with the retention member 114.

In some embodiments, the retention member 114 may be formed in an incomplete ring such that one or more gaps are formed between portions of the retention member 114. A diameter of the bore 115 along such gaps in the retention member 114 may be equivalent to the inside diameter D₃ of the bore 115. The flange 125 may be configured with arc segments configured to align with the gaps of the retention member 114 such that the plunger member 120 is freely displaced proximally and distally relative to the retention member 114 when the arc segments are aligned with the gaps, and the plunger member 120 is restricted from proximal and distal displacement when the arc segments are not aligned with the gaps.

In the illustrated embodiment, the detent 124 is positioned along the longitudinal axis of the shaft 126 relative to the retention member 114 such that the plunger member 120 is restricted from proximal displacement when the plunger member 120 is in a full distal, or fully advanced, position. Stated another way, the syringe 100 may be configured such that when the detent 124 and retention member 114 are engaged, the plunger member 120 is in a fully advanced position. This, in turn, correlates to a plunger member 120 position in which any fluid previously within the bore 115 has been expelled from the syringe 100 due to advancement of the plunger member 120.

Advancement of the detent 124 distally past the retention member 114 may be resisted by the interaction of the detent 124 and the retention member 114. For instance, as the plunger member 120 is advanced distally (from a position where the detent is proximal of the retention member 114) a distal edge of the flange 125 engages with a proximal face 114a of the retention member 114 because the diameter D₁ is greater than diameter D₂. Application of a distal force on the plunger member 120 may overcome the interference and force the detent 124 distally past the retention member 114. In some embodiments, a force between two to eight pounds may be configured to advance the detent 124 past the retention member 114.

Engagement between the detent 124 and the retention member 114 may act to restrict proximal displacement of the plunger member 120 and may be configured such that application of sufficient force may overcome the engagement. For instance, as a proximal force is applied to the plunger member 120 (from a position where the detent 124 is distal of the retention member 114 such that the detent 124 and retention member are engaged) a proximal edge of the flange 125 engages with the distal face 114b of the retention member 114 because the diameter D₁ is greater than diameter D₂. A proximally directed force on the plunger member 120 of from two to eight pounds may overcome an engagement force between the flange 125 and the retention member 114. In other words, a force of two to eight pounds applied to the plunger flange 121 by a user will allow the flange 125 to disengage from the retention member 114 and allow the plunger member 120 to be displaced proximally.

As described above, the geometry of the retention member 114 and the detent 124 may be such that the force associated with displacing the detent 124 past the retention member 114 either proximally or distally may be the same. For instance, in some embodiments a proximal or distal force between two and eight pounds may displace the detent 124 past the retention member 114. In some embodiments, the disengagement force needed to displace the plunger member 120 proximally may be greater than the disengagement force needed to displace the plunger member 120 distally. When the flange 125 engages and/or disengages from the retention member 114, an audible and/or tactile feedback may be provided to the user to indicate transitioning of the syringe 100 from a plunger locked state to a plunger unlocked state.

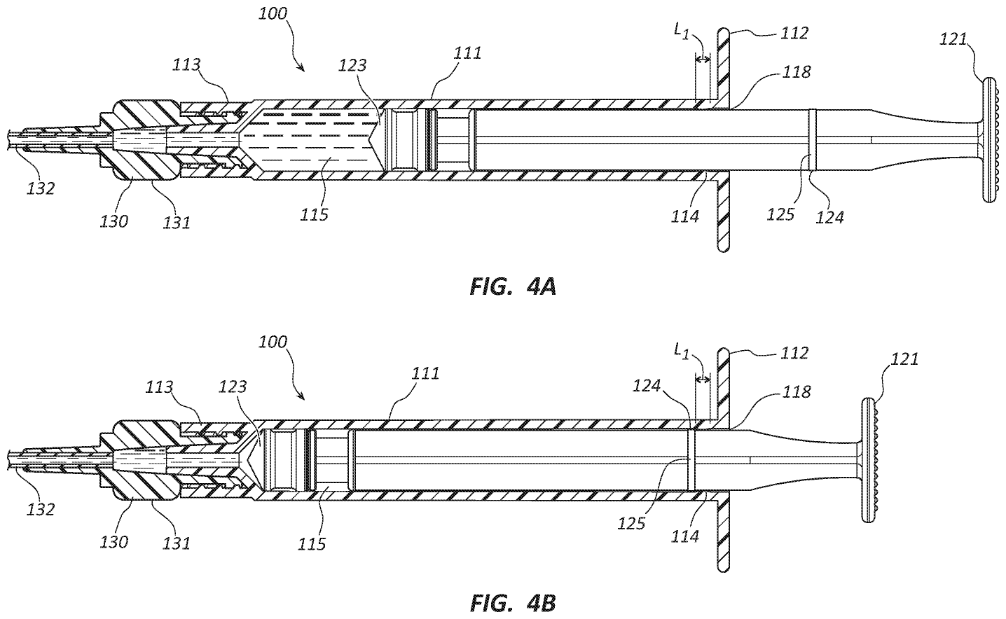

FIG. 4A depicts the syringe 100 in the plunger unlocked state where the plunger member 120 is disposed proximally such that a proximal portion of the plunger member 120 extends proximally from the barrel member 110. The detent 124 is positioned proximal to the retention member 114, and the plunger tip 123 of the plunger member 120 may be freely displaced within the bore 115 of the barrel 111. A volume of fluid and/or medicament within the bore 115 distal to the plunger tip 123 may range from about 0.25 milliliters to about 100 milliliters, including from about 0.25 milliliter to about 75 milliliters and from about 0.25 milliliter to about 60 milliliters and may be selected by the user dependent upon the volume of fluid and/or medicament required for treatment of the patient. FIG. 4B depicts the syringe in the plunger locked state where the plunger member 120 is in the full distal or advanced position, the plunger tip 123 is disposed adjacent the distal end portion 116 of the barrel 111, and the flange 125 is positioned distal of the retention member 114. In the plunger locked state, the plunger member 120 is restricted from proximal displacement and the volume of fluid and/or medicament within the bore 115 distal of the plunger tip 123 is substantially zero, meaning fluid and/or medicament within the bore 115 has been fully expelled, or a configured amount of fluid and/or medicament has been expelled, by advancement of the plunger member 120.

In use, the syringe 100 may be used to deliver a fluid and/or medicament to an intravenous (IV) infusion set, to an infusion pump, along other fluid delivery lines, directly into a patient, etc. In some procedures, the syringe may be prepared for use by coupling a fluid delivery device (e.g., a needle 130 comprising a hub 131 and a shaft 132 coupled to the hub) to the port 113 of the barrel member 110. A user may advance the plunger member 120 to the plunger locked state to evacuate any air or other material from the bore 115. As the syringe 100 transitions to the plunger locked state, the syringe 100 may be configured to provide audible and/or tactile feedback when the flange 125 engages with the retention member 114 as the plunger member 120 is displaced distally.

The needle 130 may be inserted into a fluid and/or medicament container. The plunger member 120 is retracted or displaced proximally from the plunger locked state to the plunger unlocked state, as depicted in FIG. 4A, to create a vacuum pressure within the bore 115 such that the fluid and/or medicament is drawn into the bore 115. The syringe 100 may be configured to provide audible and/or tactile feedback when the flange 125 disengages from the retention member 114 as the plunger member 120 is displaced proximally.

The needle 130 may be inserted into a reservoir of an infusion pump. In other instances, the port 113 may be directly coupled to a port of the infusion pump or coupled to any other destination device or portion of the patient's body. The plunger member 120 may be displaced distally such that the fluid and/or medicament contained within the bore 115 distal to the plunger tip 123 flows through the port 113, through the needle 130, and into the desired location. The plunger member 120 may be displaced until the plunger member 120 is in the plunger locked state, as depicted in FIG. 4B, where the volume of fluid and/or medicament contained within the bore 115 distal of the plunger tip 123 is substantially zero, or where a configured or desired amount of fluid and/or medicament has been expelled from the bore 115. Additionally, in the plunger locked state, the detent 124 is disposed distally of the retention member 114 such that the plunger member 120 is restricted from proximal displacement. The syringe 100 may be configured to provide audible and/or tactile feedback when the flange 125 engages with the retention member 114 as the plunger member 120 is displaced distally.

In some instances, a fluid backpressure may be directed into the bore 115 when the fluid and/or medicament is expelled into the desired location, such as to the reservoir of an infusion pump or into another location. This back pressure may be generated by pressure within the desired location or resistance of fluid injection into the desired location. The backpressure may apply a distally directed force to the plunger tip 123. In the absence of a detent 124, such backpressure may tend to distally displace the plunger member 120, allowing the bore 115 to partially fill with the fluid and/or medicament into the reservoir, resulting in delivery of an inaccurate quantity of fluid and/or medicament. In the illustrated embodiment of FIGS. 1-4B, the plunger member 120 is restricted from proximal displacement by the backpressure because of the engagement of the flange 125 with the retention member 114 when the plunger member 120 is in the plunger locked state. In some embodiments, the syringe 100 may be configured to withstand a backpressure of at least 25 psi without proximal displacement of the plunger member 120. In other embodiments, the geometry of the detent 124 and/or retention member 114, the size of the bore 115, or other elements may be configured such that the syringe 100 resists backpressures of at least 4 psi, at least 6 psi, at least 8 psi, or from 4 psi to 100 psi. .

It will be appreciated by one of skill in the art having the benefit of this disclosure that when the plunger member 120 is advanced to the plunger locked state, there may be a small volume of fluid and/or medicament distal of the plunger member 120 that is not ejected from the bore 115. Residual fluid may be, for instance, disposed within portions of the port 113 or there may be a small amount of fluid between the distal end of the plunger tip 123 and the distal end of the bore 115 (for instance due to tolerances in the sizes and construction of the components of the syringe 100). References herein to the locked configuration, or the fully advanced position of the plunger member 120, thus do not necessarily require complete ejection of small volumes of residual fluid or that the tolerances and manufacture of the components result in absolute evacuation of the bore 115. Furthermore, even in the presence of residual fluid in portions of the syringe 100, the plunger locked position may still correlate to a particular length of travel of the plunger member 120 between a first position and the locked position, and thus correlate to ejection of a specified amount of fluid and/or medicament. In other words, the syringe 100 may be design such that the plunger locked position correlates to a nominal fully advanced position of the plunger member 120, or correlates to a certain amount of travel of the plunger member 120, without necessarily ejecting residual fluid within the bore 115.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art may use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art, and having the benefit of this disclosure, that changes may be made to the details of the above-described embodiments without departing from the underlying principles of the disclosure herein.

1. A syringe device, comprising:
a barrel member comprising a radial inwardly extending retention member; and
a plunger member disposed at least partly within the barrel member, wherein the plunger member comprises a detent configured to restrict proximal displacement of the plunger member when exposed to a backpressure within the barrel member.

2. The syringe device of claim 1, wherein the retention member comprises a distal face and a proximal face.

3. The syringe device of claim 2, wherein the detent engages the distal face to restrict proximal displacement of the plunger member.

4. The syringe device of claim 2, wherein the plunger member is retained in a distal position when the detent engages with the distal face.

5. The syringe device of claim 4, wherein a volume of the barrel member distal to the plunger member is approximately zero when the plunger member is retained in the distal position.

6. The syringe device of claim 1, wherein an outer diameter of the detent is larger than an inner diameter of the retention member.

7. The syringe device of claim 1, wherein a distally directed force on the plunger member to overcome a first engagement force between the detent and a proximal portion of the retention member is approximately equivalent to a proximally directed force on the plunger member to overcome a second engagement force between the detent and a distal portion of the retention member.

8. The syringe device of claim 1, wherein a distally directed force on the plunger member to overcome a first engagement force between the detent and a proximal portion of the retention member is greater than a proximally directed force on the plunger member to overcome a second engagement force between the detent and a distal portion of the retention member.

9. The syringe device of claim 1, wherein the plunger member further comprises a plunger shaft, wherein a diameter of the detent is greater than a diameter of the plunger shaft.

10. The syringe device of claim 1, wherein the detent provides an audible and/or tactile feedback signal to a user when engaging with and/or disengaging from the retention member.

11. A method of transferring a fluid from a syringe device, comprising:
obtaining a syringe device comprising:
a barrel member comprising a retention member; and
a plunger member disposed at least partly within the barrel member,
wherein the plunger member comprises a detent;
proximally displacing the plunger member to fill the barrel member with a fluid;
distally displacing the plunger member to eject the fluid from the barrel member; and
engaging the detent with the retention member, wherein the plunger member is retained in a distal position when the detent engages the retention member.

12. The method of claim 11, wherein a volume of the barrel member distal to the plunger member is approximately zero when the plunger member is retained in the distal position.

13. The method of claim 11, wherein proximally displacing the plunger member comprises engaging the detent with the retention member.

14. The method of claim 11, further comprising retaining the plunger member in the distal position when the plunger member is exposed to a backpressure.

15. The method of claim 11, further comprising detecting an audible and/or tactile feedback when the detent engages the retention member.

16. A fluid transfer system, comprising:
a syringe device, comprising:
a barrel member, comprising a retention member; and
a plunger member, comprising a detent; and
a fluid delivery device in fluid communication with the syringe device.

17. The fluid transfer system of claim 16, wherein the detent is configured to engage with the retention member to retain the plunger member in a distal position within the barrel member.

18. The fluid transfer system of claim 16,
wherein a fluid is dispelled from the barrel member through the fluid transfer device when the plunger member is displaced distally within the barrel member, and
wherein the plunger member is configured to be retained in a distal position within the barrel member by engagement of the retention member with the detent.

19. The fluid transfer system of claim 18, wherein a fluid volume within the barrel member is approximately zero when the barrel member is in the distal position.

20. The fluid transfer system of claim 16, wherein the fluid delivery device comprises a female Luer fitting configured to couple with a male Luer fitting of the syringe device.
